(19) United States
(12) Patent Application Publication
(10) Pub. No.: US 2026/0356985 A1
(43) Pub. Date: Nov. 12, 2026

(54) METHODS OF PREPARING ANTIBODY-POLYNUCLEOTIDE CONJUGATES

(71) Applicant: Janssen Pharmaceutica NV, Beerse (BE)

(72) Inventors: Shalom Goldberg, Spring House, PA (US); Rebecca Wissner, Spring House, PA (US); Michael McCauley, Jr., Spring House, PA (US)

(21) Appl. No.: 19/140,495

(22) PCT Filed: Dec. 14, 2023
(86) PCT No.: PCT/IB2023/062719

§ 371 Date: Jun. 18, 2025

Related U.S. Application Data
(60) Provisional application No. 63/434,517, filed on Dec. 22, 2022.
(60) Provisional application No. 63/434,532, filed on Dec. 22, 2022.

(51) Int. Cl. C07K 19/00

(52) U.S. Cl. CPC C07K 19/00

(57) ABSTRACT

Provided herein are methods of forming an antibody-polynucleotide conjugate having an antibody-polynucleotide ratio that is about 1:1 and compositions of the same. These methods allow for efficient generation of antibody-polynucleotides conjugates with precise stoichiometries. Further, these conjugates have improved PK properties and are easier to manufacture compared to other methods.

### CROSS-REFERENCE

This application claims priority to U.S. Provisional Application No. 63/434,517, filed Dec. 22, 2023, and U.S. Provisional Application No. 63/434,532, filed Dec. 22, 2023, the contents of which are incorporated by reference in their entireties.

### INCORPORATION BY REFERENCE

All publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls.

### BACKGROUND

Exogenous polynucleotides can exert diverse effects on cellular physiology which can be utilized for a variety of purposes. In particular, delivery of polynucleotides (such as interfering RNAs) has emerged as a promising therapeutic strategy for the treatment of various diseases. However, polynucleotides are typically too large, hydrophilic, and negatively charged to spontaneously cross the cell membrane. Moreover, polynucleotides may be susceptible to degradation by extracellular and intracellular nucleases.

Short interfering RNAs (siRNAs) are a class of double-stranded nucleotides that can drive the sequence-specific degradation of target mRNAs by engaging the RNA-induced silencing complex (RISC). These molecules typically suffer from being too large, hydrophilic, and negatively charged to spontaneously cross the cell membrane. Further, unmodified siRNAs are also susceptible to degradation.

### SUMMARY

Disclosed herein are methods for forming an antibody-polynucleotide conjugate comprising contacting an antibody comprising two reactive bioorthogonal groups, and a polynucleotide comprising a bifurcated linker that comprises two reactive groups, wherein the reactive groups on the bifurcated linker selectively interact with the biorthogonal groups to form a conjugate having an antibody-polynucleotide ratio that is about 1:1.

Disclosed herein are methods of forming an antibody-polynucleotide conjugate comprising contacting an antibody and a polynucleotide, wherein each antibody heavy chain constant region comprises an azide group, and the polynucleotide comprises a bifurcated linker that comprises two alkyne groups, wherein each azide group reacts with an alkyne group to form a conjugate having an antibody-polynucleotide ratio that is about 1:1.

Disclosed herein are compositions comprising a Mal-PEG(X)-bis-PEG(X)-BCN or a Mal-PEG(X)-bis-PEG(X)-DBCO conjugated to a polynucleotide.

Disclosed herein are polynucleotide-conjugated antibodies comprising a polynucleotide attached to a bifurcated linker, and an antibody comprising two heavy chain constant regions that each comprise a bioorthogonal group, wherein the bifurcated linker is attached to the bioorthogonal groups of each heavy chain constant region.

Disclosed herein are methods of forming an antibody-polynucleotide conjugate comprising contacting an antibody comprising a F405L substitution and a R409K substitution on one heavy chain constant region and an azide group on the other heavy chain constant region, and a polynucleotide to form a conjugate having an antibody-polynucleotide ratio that is about 1:1.

Disclosed herein are methods of promoting Fab arm exchange comprising contacting a first antibody comprising a F405L substitution and a R409K substitution, and a second antibody that does not have the F405L substitution and the R409K substitution and that comprises an azide group on one or both of the antibody heavy chain constant regions to form an antibody comprising the F405L substitution and the R409K substitution on one heavy chain constant region and the azide group on the other heavy chain constant region.

### BRIEF DESCRIPTION OF THE DRAWINGS

The summary, as well as the following detailed description, is further understood when read in conjunction with the appended drawings. For the purpose of illustrating the disclosed methods, conjugated polynucleotide molecules (such as siRNA molecules), and polynucleotide-conjugated antibodies, the drawings show exemplary embodiments of the methods and compositions; however, the methods and compositions are not limited to the specific embodiments disclosed. In the drawings:

FIG. 1 illustrates an exemplary scheme depicting the generation of 1:1 mAb-polynucleotide conjugates using an siRNA as an exemplary polynucleotide.

FIG. 2 illustrates an exemplary synthesis reaction for generating a Mal-bis-BCN-polynucleotide. In this case, a Mal-bis-BCN-polynucleotideCTNNB1 siRNA was generated.

FIG. 3 illustrates an SDS-PAGE analysis of reduced PSMB127-siRNA conjugates.

FIG. 4 illustrates an SDS-PAGE analysis of reduced Trastuzumab-siRNA conjugates.

FIG. 5A and FIG. 5B illustrate analytical SEC chromatograms (280 nm absorbance trace) corresponding to 1:1 PSMB 127-siRNA conjugates generated by controlled Fab arm exchange (cFAE) (FIG. 5A) and by the stitching method using azido-modified antibodies and Mal-bis-BCN siRNAs (FIG. 5B).

FIG. 6A, FIG. 6B, FIG. 6C, and FIG. 6D illustrate analytical SEC chromatograms (280 nm absorbance trace) corresponding to 1:1 Trastuzumab-siRNA conjugates generated by chromatographic isolation (FIG. 6A and FIG. 6C) and by the stitching method using azido-modified antibodies and Mal-bis-BCN siRNAs (FIG. 6B and FIG. 6D).

FIG. 7 illustrates CTNNB1 mRNA knockdown following treatment of PSMA-expressing HEK-293T cells with PSMB127 antibody:CTNNB1-siRNA conjugates.

FIG. 8 illustrates CTNNB1 mRNA knockdown following treatment of HCC1954 cells with Trastuzumab: CTNNB1-siRNA conjugates.

FIG. 9 illustrates the pharmacokinetics of Trastuzumab-siRNA conjugates in C57BL/6 mice over time.

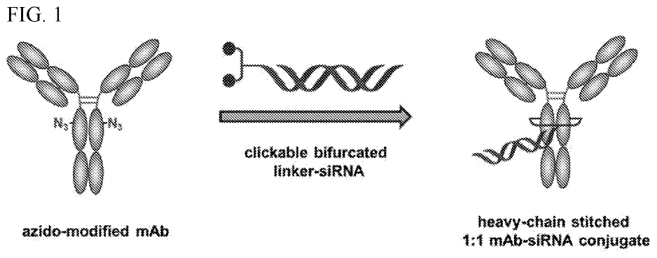

### DETAILED DESCRIPTION

The disclosed methods, conjugated polynucleotide molecules (e.g., siRNA conjugated molecules), and polynucleotide-conjugated antibodies may be understood more readily by reference to the following detailed description taken in connection with the accompanying figures, which form a part of this disclosure. The disclosed methods and compositions are not limited to the specific methods and compositions described and/or shown herein, and the terminology used herein is for the purpose of describing particular embodiments by way of example only and is not intended to be limiting of the claimed methods and compositions.

Unless specifically stated otherwise, any description as to a possible mechanism or mode of action or reason for improvement is meant to be illustrative only, and the disclosed methods and compositions are not to be constrained by the correctness or incorrectness of any such suggested mechanism or mode of action or reason for improvement.

Throughout this text, the descriptions refer to compositions and methods of using said compositions. Where the disclosure describes or claims a feature or embodiment associated with a composition, such a feature or embodiment is equally applicable to the methods of using said composition. Likewise, where the disclosure describes or claims a feature or embodiment associated with a method of using a composition, such a feature or embodiment is equally applicable to the composition.

Certain features of the disclosed methods and compositions which are, for clarity, described herein in the context of separate embodiments, may also be provided in combination in a single embodiment. Conversely, various features of the disclosed methods and compositions that are, for brevity, described in the context of a single embodiment, may also be provided separately or in any subcombination.

As used herein, the singular forms “a,” “an,” and “the” include the plural.

Various terms relating to aspects of the description are used throughout the specification and claims. Such terms are to be given their ordinary meaning in the art unless otherwise indicated. Other specifically defined terms are to be construed in a manner consistent with the definitions provided herein.

The term “comprising” is intended to include examples encompassed by the terms “consisting essentially of” and “consisting of”; similarly, the term “consisting essentially of” is intended to include examples encompassed by the term “consisting of.”

The term “about” when used in reference to numerical ranges, cutoffs, or specific values is used to indicate that the recited values may vary by up to as much as 10% from the listed value. Thus, the term “about” is used to encompass variations of +10% or less, variations of +5% or less, variations of +1% or less, variations of +0.5% or less, or variations of +0.1% or less from the specified value.

As used herein, “administering” and similar terms indicate a procedure by which an antibody-polynucleotide conjugate is exposed to target cells in vitro, or is injected into a subject such that target cells, tissues, or segments of the body of the subject are contacted with the antibody-polynucleotide conjugate.

The term “subject” as used herein is intended to mean any animal, in particular, mammals. Although treatment of in mice with an antibody-polynucleotide conjugate is exemplified herein, any type of mammal can be treated using the disclosed methods. Any type of cell or mammal can be treated using the disclosed methods. Thus, the methods are applicable to human and nonhuman animals, although preferably used with mice and humans, and most preferably with humans. “Subject” and “patient” are used interchangeably herein. The subjects herein can be a subject that is in need thereof.

The term “antibody,” and like terms is meant in a broad sense and includes immunoglobulin molecules including, monoclonal antibodies, antibody fragments, bispecific or multispecific antibodies, dimeric, tetrameric or multimeric antibodies, and single chain antibodies. Immunoglobulins can be assigned to five major classes, namely IgA, IgD, IgE, IgG, and IgM, depending on the heavy chain constant domain amino acid sequence. IgA and IgG are further sub-classified as the isotypes IgA1, IgA2, IgG1, IgG2, IgG3, and IgG4. Antibody light chains of any vertebrate species can be assigned to one of two clearly distinct types, namely kappa (κ) and lambda (λ), based on the amino acid sequences of their constant domains.

“Stitched,” as used herein, refers to antibody (Ab):polynucleotide conjugates, such as an Ab:siRNA conjugate, wherein the Ab has attached thereto a bifurcated linker, such that the bifurcated linker is attached to two different protein chains of the Ab via a biorthogonal group (such as one end of the bifurcated linker being attached to a biorthogonal group on one heavy chain constant region and the other end of the bifurcated linker being attached to a biorthogonal group on the other heavy chain constant region) and whereby the bifurcated linker is attached to a polynucleotide or payload, such as an siRNA.

One approach for polynucleotide delivery is to chemically conjugate the nucleic acid to a bioactive targeting moiety such as a monoclonal antibody (mAb). mAbs exhibit long serum half-lives and selectively bind their target antigens with high affinity, rendering cell-type specific targeting possible. mAbs that target internalizing cell-surface receptors can be further exploited to deliver conjugated payloads, including polynucleotides such as siRNAs, through the endo/lysosomal pathway. For an siRNA, for example, to exert its gene silencing effect, it must ultimately be delivered to the cytosol to engage the RISC complex. Multiple hurdles need to be overcome to improve a polynucleotide's in vivo efficacy, such as inefficient endo/lysosomal escape, the influence that a polynucleotide can have on the serum half-life of the mAb, and the challenge of conjugating the polynucleotide to the mAb in the proper ratio for efficient delivery and function.

Disclosed herein are methods of forming an antibody-polynucleotide conjugate. The methods can comprise contacting an antibody comprising two reactive bioorthogonal groups and an polynucleotide comprising a bifurcated linker that comprises two reactive groups wherein the reactive groups on the bifurcated linker selectively interact with the biorthogonal groups to form a conjugate having an antibody-polynucleotide ratio that is about 1:1. Bioorthogonal groups can be chemical moieties that take part in chemical reactions that are selective enough to proceed in complex biological environments without being impacted by the presence of amino acids, nucleic acids, etc.

The methods can comprise contacting an antibody and polynucleotide, wherein each antibody heavy chain constant region comprises an azide group, and the polynucleotide comprises a bifurcated linker that comprises two alkyne groups, wherein each azide groups reacts with an alkyne group to form a conjugate having an antibody-polynucleotide ratio that is about 1:1.

The polynucleotide conjugated to the antibody can be DNA or RNA, or analogs thereof. For instance, in some embodiments, the DNA or the RNA is a phosphorodiamidate morpholino oligomer (PMO). Suitable RNAs include siRNA, miRNA, gRNA, shRNA, or an antisense oligonucleotide. The polynucleotide may be coding or noncoding. The polynucleotide may comprise DNA or RNA nucleotide modifications, such as methylation. The polynucleotide may be single stranded or double stranded. The polynucleotide may be linear or circular. The polynucleotide may be in various secondary or tertiary conformations due to internal base pairing.

The reactive biorthogonal groups can be located within the antibody's heavy chain constant region. In some embodiments, one antibody heavy chain constant region comprises one reactive biorthogonal group and the other antibody heavy chain constant region comprises the other reactive biorthogonal group. Each of the two reactive biorthogonal groups can comprise an azide group. Suitable azide groups include, but are not limited to, 3-azidopropylamine, N-azidoacetylgalactosamine, 6-azido GalNAc, azido-butylamine, azidepentylamine, and azide-PEG3-amine. In some embodiments, at least one of the azide groups can be 3-azidopropylamine. In some embodiments, at least one of the azide groups can be N-azidoacetylgalactosamine. In some embodiments, at least one of the azide groups can be 6-azido GalNAc. In some embodiments, at least one of the azide groups can be azido-butylamine. In some embodiments, at least one of the azide groups can be azidepentylamine. In some embodiments, at least one of the azide groups can be azide-PEG3-amine. Each antibody heavy chain can comprise the same bioorthogonal group. In some embodiments, each antibody heavy chain can comprise 3-azidopropylamine. In some embodiments, each antibody heavy chain can comprise N-azidoacetylgalactosamine. In some embodiments, each antibody heavy chain can comprise 6-azido GalNAc. In some embodiments, each antibody heavy chain can comprise azido-butylamine. In some embodiments, each antibody heavy chain can comprise azidepentylamine. In some embodiments, each antibody heavy chain can comprise azide-PEG3-amine. Alternatively, each antibody heavy chain can comprise a different bioorthogonal group. For example, one antibody heavy chain can comprise 3-azidopropylamine and one antibody heavy chain can comprise N-azidoacetylgalactosamine.

The CH2 region of the antibody can bear the reactive bioorthogonal group. Suitable antibody heavy chain positions for the bioorthogonal group include Q295 and N297. Suitable azides at position Q295 include 3-azidopropylamine, azido-butylamine, azidepentylamine, and azide-PEG3-amine. In some embodiments, the antibody has 3-azidopropylamine at amino acid position Q295. One or both Q295 positions can bear 3-azidopropylamine. Suitable azides at position N297 include N-azidoacetylgalactosamine and 6-azido GalNAc. In some embodiments, the antibody has N-azidoacetylgalactosamine at amino acid position N297. One or both N297 positions can bear N-azidoacetylgalactosamine.

Suitable bifurcated linkers include those capable of reacting with the bioorthogonal group present on the antibody heavy chains. For example, in the case of antibodies comprising bioorthogonal groups with azide groups, the bifurcated linker can comprise alkyne reactive groups. The alkyne group can comprise a cyclooctyne. The bifurcated linker can include two reactive groups, wherein each group reacts with a bioorthogonal group on the antibody heavy chain. The bifurcated linker can be a Mal-PEG(X)-bis-PEG(X)-BCN linker or a Mal-PEG(X)-bis-PEG(X)-DBCO linker, wherein (X) represents the number of PEG units. The number of PEG units in the general formulas Mal-PEG(X)-bis-PEG(X)-BCN linker and Mal-PEG(X)-bis-PEG(X)-DBCO can be about 1 to 10. In some embodiments, the bifurcated linker is a Mal-PEG2-bis-PEG3-BCN.

Disclosed herein are compositions comprising a Mal-PEG(X)-bis-PEG(X)-BCN or a Mal-PEG(X)-bis-PEG(X)-DBCO conjugated to a polynucleotide, wherein (X) represents the number of PEG units. The number of PEG units in the general formulas Mal-PEG(X)-bis-PEG(X)-BCN linker and Mal-PEG(X)-bis-PEG(X)-DBCO can be about 1 to 10. In some embodiments, Mal-PEG(X)-bis-PEG(X)-BCN is conjugated to the polynucleotide. In some embodiments, Mal-PEG(X)-bis-PEG(X)-DBCO is conjugated to the polynucleotide. In some embodiments, the bifurcated linker is a Mal-PEG2-bis-PEG3-BCN. The Mal-PEG(X)-bis-PEG(X)-BCN or Mal-PEG(X)-bis-PEG(X)-DBCO can be attached to the polynucleotide by a dihexyldisulfide linker. The Mal-PEG(X)-bis-PEG(X)-BCN or Mal-PEG(X)-bis-PEG(X)-DBCO can be attached to the 5′ end of the polynucleotide. In some embodiments, the Mal-PEG(X)-bis-PEG(X)-BCN or Mal-PEG(X)-bis-PEG(X)-DBCO is attached to the 5′ end of the sense strand of the polynucleotide. The Mal-PEG2-bis-PEG3-BCN conjugated polynucleotide molecule can be reacted with an antibody that comprises bioorthogonal groups to form a polynucleotide-conjugated antibody as described herein.

In the herein described compositions comprising a Mal-PEG(X)-bis-PEG(X)-BCN or a Mal-PEG(X)-bis-PEG(X)-DBCO conjugated to a polynucleotide, the polynucleotide can be DNA or RNA, or analogs thereof. For instance, in some embodiments, the DNA or the RNA is a PMO. Suitable RNAs include siRNA, miRNA, gRNA, shRNA, or an antisense oligonucleotide. The polynucleotide may be coding or noncoding. The polynucleotide may comprise DNA or RNA nucleotide modifications, such as methylation. The polynucleotide may be single stranded or double stranded. The polynucleotide may be linear or circular. The polynucleotide may be in various secondary or tertiary conformations due to internal base pairing.

Disclosed herein are polynucleotide-conjugated antibodies comprising a polynucleotide attached to a bifurcated linker and an antibody comprising two heavy chain constant regions that each comprise a bioorthogonal group, wherein the bifurcated linker is attached with the bioorthogonal group of each heavy chain constant region. The bifurcated linker can be a Mal-PEG(X)-bis-PEG(X)-BCN or a Mal-PEG(X)-bis-PEG(X)-DBCO, wherein (X) represents the number of PEG units. The number of PEG units in the general formulas Mal-PEG(X)-bis-PEG(X)-BCN linker and Mal-PEG(X)-bis-PEG(X)-DBCO can be about 1 to 10. The bifurcated linker can be Mal-PEG2-bis-PEG3-BCN. The polynucleotide can be conjugated to the antibody by a triazole. The polynucleotide-conjugated antibody can be generated by the methods described herein.

In the herein described polynucleotide-conjugated antibodies, the polynucleotide conjugated to the antibody can be DNA or RNA, or analogs thereof. For instance, in some embodiments, the DNA or the RNA is a PMO. Suitable RNAs include siRNA, miRNA, gRNA, shRNA, or an antisense oligonucleotide. The polynucleotide may be coding or noncoding. The polynucleotide may comprise DNA or RNA nucleotide modifications, such as methylation. The polynucleotide may be single stranded or double stranded. The polynucleotide may be linear or circular. The polynucleotide may be in various secondary or tertiary conformations due to internal base pairing.

Described herein are methods of delivering a polynucleotide to a target, comprising administering any of the herein disclosed polynucleotide-conjugated antibodies. Any of the herein described polynucleotides can be delivered to a target via the described polynucleotide-conjugated antibodies. Polynucleotides can be delivered for a wide variety of reasons such as genomic modification, introduction of exogenous polynucleotides for use by the cellular machinery, or induction of cellular responses that are sensitive to the delivered polynucleotide. Suitable polynucleotides delivered for genomic modification include, but are not limited to, gRNAs and insertion cassettes. Suitable polynucleotides delivered for use by the cellular machinery, include but are not limited to, mRNA, tRNA, miRNA, or coding DNA. Suitable polynucleotides delivered for induction of cellular responses, such as the immune response, include single stranded RNA, double stranded RNA, and CpG containing DNA.

Described herein are methods of gene silencing or reducing gene expression comprising administering any of the herein disclosed polynucleotide-conjugated antibodies. Polynucleotides capable of promoting gene silencing or reducing gene expression include those that reduce the transcription of translation of mRNAs. Coding or noncoding RNAs can be targeting for gene silencing or a reduction in gene expression. In some embodiments, the RNA to be silenced is an mRNA. Suitable polynucleotides administered for gene silencing or reducing gene expression include, but are not limited to, siRNA, shRNA, and PMO polynucleotides.

The polynucleotide-conjugated antibody can be administered to a subject in need thereof. The subject can be a human subject or a non-human subject. The subject can be a subject in need thereof. The polynucleotide-conjugated antibody can be administered to cells in vitro.

Disclosed herein are methods of forming an antibody-polynucleotide conjugate, the methods comprising contacting an antibody comprising a F405L substitution and a R409K substitution, according to EU numbering index, on one heavy chain constant region and an azide group on the other heavy chain constant region and a polynucleotide to form a conjugate having an antibody-polynucleotide ratio that is about 1:1.

In some embodiments, the methods comprise, prior to contacting the antibody and the polynucleotide, contacting an antibody comprising a F405L substitution and an R409K substitution, according to EU numbering index, and an antibody that does not have the F405L substitution and the R409K substitution and that comprises an azide group on one or both of the antibody heavy chain constant regions to promote Fab arm exchange and the formation of an antibody comprising the F405L substitution and the R409K substitution on one heavy chain constant region and the azide group on the other heavy chain constant region.

Disclosed herein are methods of promoting Fab arm exchange comprising contacting a first antibody comprising a F405L substitution and a R409K substitution, according to EU numbering index, and a second antibody that does not have the F405L substitution and the R409K substitution and that comprises an azide group on one or both of the antibody heavy chain constant regions, to form an antibody comprising the F405L substitution and the R409K substitution on one heavy chain constant region and the azide group on the other heavy chain constant region.

As used herein, “Fab arm exchange” (or half molecule exchange) refers to the process by which half antibodies (a light chain and heavy chain pair) associate to form intact antibodies (two light chain and heavy chain pairs). The Fab arm exchange reaction is the result of a disulfide-bond isomerization reaction and dissociation-association of CH3 domains. The heavy-chain disulfide bonds in the hinge regions of the antibodies are reduced, and the resulting free cysteines of the antibody can form an inter heavy-chain disulfide bond with cysteine residues of another antibody. CH3 domains of the antibodies simultaneously release and reform by dissociation-association. Fab arm exchange can be used to generate heterodimers (such as bispecific antibodies) by introducing amino acid substitutions (such as a F405L substitution and an R409K substitution) into the heavy chain CH3 domains of mono-specific bivalent antibodies to thereby favor formation of the heterodimer. Following dissociation of the mono-specific bivalent antibodies, for example, antibody halves containing the amino acid substitutions will preferably associate with antibody halves not having the amino acid substitutions to thereby drive formation of the heterodimer. Fab arm exchange is described in, for example, U.S. Pub. No. US20220267438.

In some embodiments, the antibody that does not have the F405L substitution and the R409K substitution comprises an azide group on one of the heavy chain constant regions. In some embodiments, the antibody that does not have the F405L substitution and the R409K substitution comprises azide groups on both of the heavy chain constant regions. The CH2 region of the antibody can bear the azide group. Suitable azide groups on one or both of the antibody heavy chain constant regions include, but are not limited to, 3-azidopropylamine, N-azidoacetylgalactosamine, 6-azido GalNAc, azido-butylamine, azidepentylamine, and azide-PEG3-amine. Suitable antibody positions for the azide group include Q295 and N297. Suitable azides at position Q295 include 3-azidopropylamine, azido-butylamine, azidepentylamine, and azide-PEG3-amine. In some embodiments, the antibody has 3-azidopropylamine at amino acid position Q295. One or both Q295 positions can bear 3-azidopropylamine. Suitable azides at position N297 include N-azidoacetylgalactosamine and 6-azido GalNAc. In some embodiments, the antibody has N-azidoacetylgalactosamine at amino acid position N297. One or both N297 positions can bear N-azidoacetylgalactosamine.

Polynucleotides suitable for contact with the antibody comprising the F405L substitution and the R409K substitution on one heavy chain constant region and the azide group on the other heavy chain constant region include those capable of reacting with the azide group. The polynucleotide can comprise one or more reactive groups capable of reacting with the azide group. For example, the polynucleotide can comprise one or more alkyne reactive groups capable of reacting with the azide. In some embodiments, the alkyne can comprise a cyclooctyne. In some embodiments, the alkene reactive group is dibenzocyclooctyne (DBCO).

The antibody comprising the F405L substitution and the R409K substitution can be an IgG4 antibody. The antibody that does not have the F405L substitution and the R409K substitution comprising the azide group(s) can be an IgG4. In some embodiments, both the antibody comprising the F405L substitution and the R409K substitution and the antibody that does not have the F405L substitution and the R409K substitution comprising the azide group(s) can be an IgG4. The antibody comprising the F405L substitution and the R409K substitution can be monospecific, bispecific, or multispecific. The antibody that does not have the F405L substitution and the R409K substitution comprising the azide group(s) can be monospecific, bispecific, or multispecific. In some embodiments the antibody comprising the F405L substitution and the R409K substitution binds to the same antigen as the antibody that does not have the F405L substitution and the R409K substitution comprising the azide group(s). In some embodiments, the antibody comprising the F405L substitution and the R409K substitution binds to a different antigen as the antibody that does not have the F405L substitution and the R409K substitution comprising the azide group(s). The antibody comprising the F405L substitution and the R409K substitution on one heavy chain constant region and the azide group on the other heavy chain constant region can be monospecific, bispecific, or multispecific.

The antibody-polynucleotide conjugate described throughout has similar or improved biophysical stability profiles compared to non-conjugated antibodies and/or polynucleotides. For example, thermal stability of the antibody-polynucleotide conjugate is retained compared to the parental antibody and synthetic intermediates such as apa and GlaNAz. Differential scanning fluorometry can be used to analyze biophysical stability of the conjugates. Further analysis of the antibody-polynucleotide conjugates (by e.g., analytical size exclusion chromatography) demonstrates that the conjugates are predominantly monomeric and intact.

The antibody-polynucleotide conjugates also induce efficient gene silencing and is at least equivalent or better than an antibody-polynucleotide conjugates generated by other methods. For example, other methods of generating antibody-polynucleotide conjugates include chromatographic isolation or Fab-arm exchange.

Importantly, the antibody-polynucleotide conjugate is produced by the methods described throughout can lead to an overall yield of greater than other known methods such as chromatographic isolation. For example, chromatographic isolation typically produces antibody-polynucleotide conjugate yields at around 30% at best. The methods described throughout produce antibody-polynucleotide conjugates at an overall yield of greater than about 50%. For example, in some cases the yield can be between about 50% to about 100%. In other cases, the yield can be between about 55% to about 85%. In other cases, the yield can be between about 55% to about 75%. In other cases, the yield can be between about 55% to about 70%.

The antibody-polynucleotide conjugates produced by the methods described throughout have an improved PK profile (e.g., plasma concentration) compared to an antibody-polynucleotide conjugate that has a 1:2 ratio (even when produced by the same methods described throughout).

The method described throughout ultimately leads to an easier and more efficient method of producing antibody-polynucleotide conjugates.

### EXAMPLES

The following examples are provided to further describe some of the embodiments disclosed herein. The examples are intended to illustrate, not to limit, the disclosed embodiments.

### Example 1

Preparation of Azido-Modified mAbs.

Microbial transglutaminase (MTG)-catalyzed conjugation of 3-azidopropylamine to mAb position Q295. All mAbs were expressed in CHO cells and purified by protein A chromatography using standard methods. mAbs (PSMB127 and Trastuzumab) were fully deglycosylated using Rapid PNGase F (P0710, New England Biolabs). Briefly, Rapid PNGase F was added to each mAb (5 μL/mg mAb) and the reaction was incubated overnight at 37 °C. The deglycosylated mAbs (1 mg/mL) were reacted with 100 equivalents of 3-azidopropylamine (3-apa) and MTG (Activa TI, 20% w/v) in Dulbecco's phosphate buffered saline (DPBS) for 4-6 hours at 37 °C. Reaction progress was monitored by mass spectrometry. The apa-modified mAbs were purified by protein A affinity chromatography and analyzed by mass spectrometry and analytical size-exclusion chromatography. 3-azidopropylamine was conjugated to amino acid position Q295 using this procedure.

Site-specific azido-modification of mAb Fc glycans. mAbs (PSMB127 and Trastuzumab) were deglycosylated with EndoS2 (A0-GL8-020, GlycINATOR LE, Genovis), an enzyme that hydrolyzes the β1,4 linkage between the core N-Acetylglucosamine (GlcNAc) residues of the Fc glycans, leaving the innermost GlcNAc resides intact (one per heavy chain). GlycINATOR LE was added to each mAb (1-5 L/mg mAb) and incubated overnight at 37 °C. The core GlcNAc residues were subsequently conjugated to N-azidoacetylgalactosamine (GalNAz) using components from the SiteClick™ antibody azido modification system kit (S10901, Thermo Fisher Scientific). Briefly, the deglycosylated mAbs were exchanged into tris buffered saline (TBS, pH 7.4) and concentrated to 6 mgs/mL. The mAbs were then reacted with the provided GalT enzyme (60 μL/mg mAb) in the presence of UDP-GalNAz (2.5 mM) overnight at 30 °C. Reaction progress was monitored by mass spectrometry. The GalNAz-modified mAbs were purified by diafiltration and analyzed by mass spectrometry and analytical size exclusion chromatography. N-azidoacetylgalactosamine (GalNAz) was conjugated to amino acid position N297 using this procedure.

Preparation of siRNA Having a Bifurcated Linker

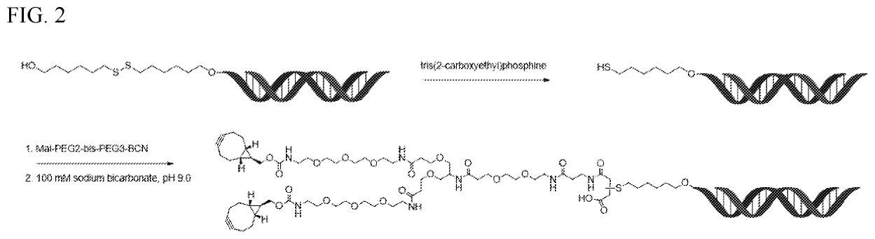

Preparation of Mal-bis-BCN-CTNNB1 siRNA. A variant of a chemically stabilized siRNA targeting CTNNB1 containing a 5′-dihexyldisulfide linker on the sense strand was prepared by solid-phase synthesis. The disulfide protected siRNA was dissolved in water and deprotected using 15 equivalents of tris(2-carboxyethyl)phosphine (TCEP) overnight at 37 °C. The deprotected siRNA was exchanged into 20 mM Tris, pH 8.0 by diafiltration and subsequently reacted with 10 equivalents of Mal-PEG2-bis-PEG3-BCN (“Mal-bis-BCN”) (CP-2097, Conju-Probe) to yield the Mal-bis-BCN-CTNNB1 siRNA. If the succinimide moiety of a maleimide-thiol conjugate is hydrolyzed, the ring-opened product is stabilized toward cleavage in vivo as it is no longer susceptible to thiol exchange reactions. Following conjugation of Mal-PEG2-bis-PEG3-BCN, the reaction pH was adjusted to 9.0 by adding sodium bicarbonate to a final concentration of 100 mM to drive the formation of the ring-opened product (FIG. 2). The reaction was incubated overnight at 4 °C and the extent of maleimide ring-opening was monitored by mass spectrometry. Upon complete conversion to the maleimide ring-opened product, the Mal-bis-BCN-CTNNB1 siRNA was purified by anion exchange chromatography, dialyzed into DPBS, and stored at −20 °C until further use. Using this method, the bifurcated linker (Mal-bis-BCN) was conjugated to the siRNA at the 5′ end at an inverted abasic residue (i.e. at the 5′ OH of the abasic group). The modification occurred on the sense strand.

Preparation of mAb-siRNA Conjugates

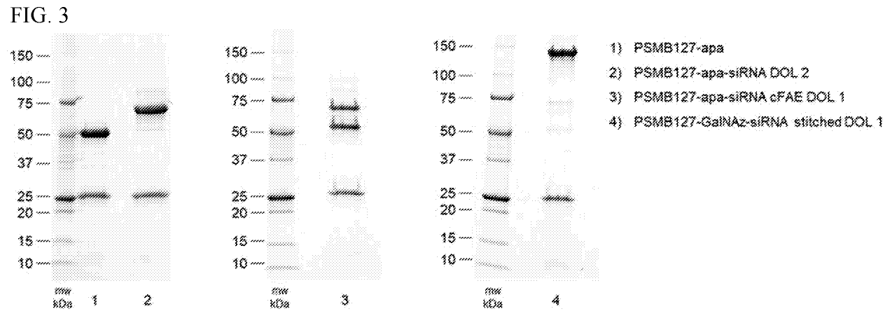

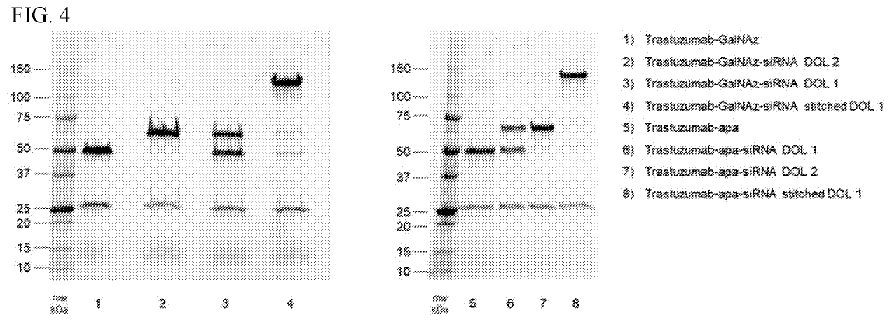

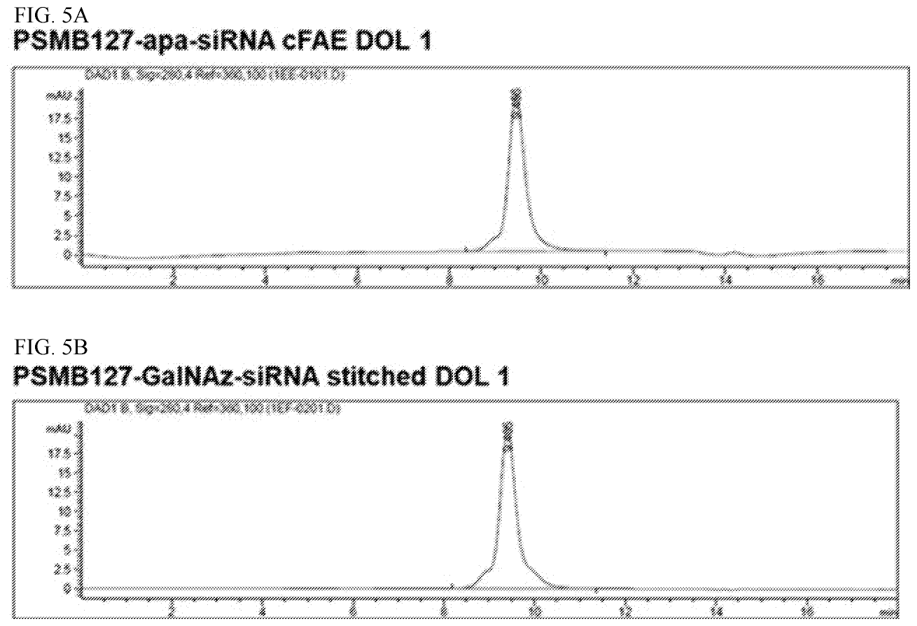

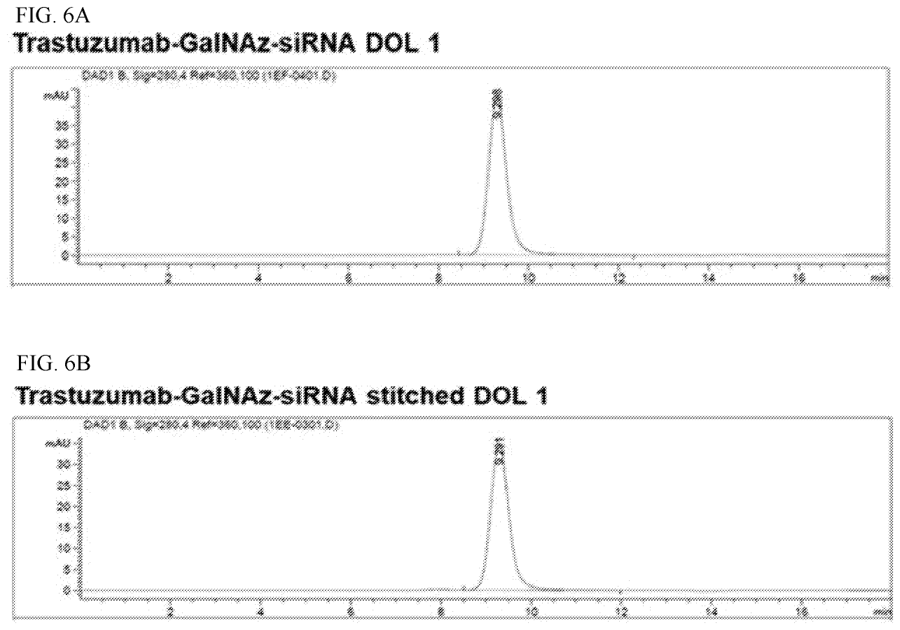

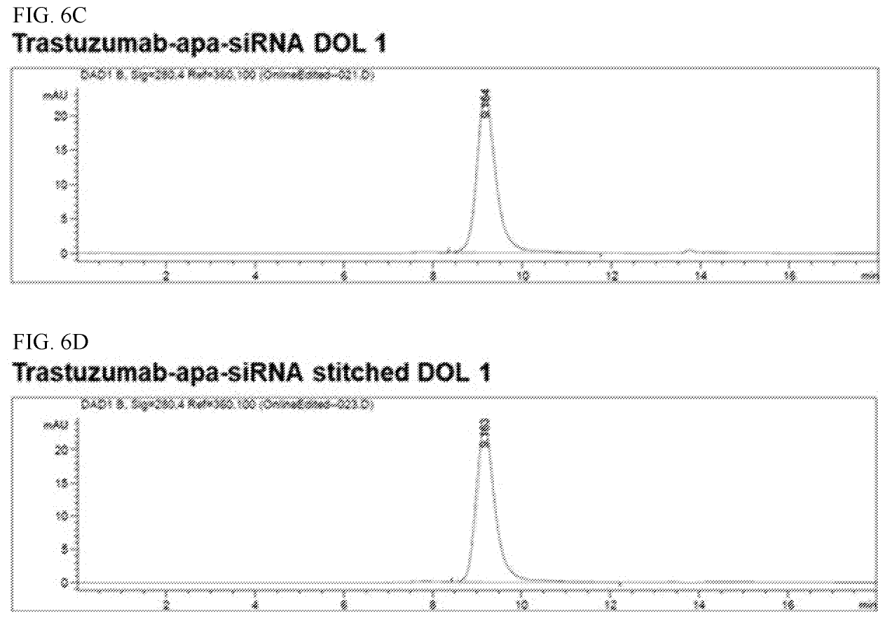

Preparation of “stitched” 1:1 mAb-siRNA conjugates using azido-modified mAbs and Mal-bis-BCN siRNA. Heavy chain “stitched” 1:1 mAb-siRNA conjugates were prepared through strain-promoted azide-alkyne cycloaddition chemistry (copper-free click chemistry) by reacting the azido-modified mAbs (PSMB127-GalNAz, Trastuzumab-apa, and Trastuzumab-GalNAz) with 2-5 equivalents of Mal-bis-BCN-CTNNB1 siRNA overnight at 37 °C. Reaction progress was monitored by SDS-PAGE analysis. The mAb-siRNA conjugates were purified by size exclusion chromatography or anion-exchange chromatography over a TSK-Gel™ BioAssist™ Q column. 1:1 mAb-siRNA conjugates (PSMB127-GalNAz-siRNA stitched DOL 1, Trastuzumab-GalNAz-siRNA stitched DOL 1, and Trastuzumab-apa-siRNA stitched DOL 1) were analyzed by reduced SDS-PAGE and analytical size exclusion chromatography. SDS-PAGE analysis of the reduced mAbs confirmed formation of the covalently linked heavy-chain dimer in each case, as evidenced by the presence of a protein band that runs between the 100 and 150 kDa molecular weight markers (FIG. 3 and FIG. 4). Analytical size exclusion chromatography confirmed that the mAbs were monomeric and intact (FIG. 5A, FIG. 5B, FIG. 6A, FIG. 6B, FIG. 6C, and FIG. 6D). The retention time of the 1:1 mAb-siRNA conjugates was equivalent to those observed for the 1:1 mAb-siRNA conjugates prepared by other methods (cFAE or chromatographic isolation method). The obtained yields of the 1:1 conjugates (from each respective azido-mAb intermediate) ranged from 55-70%, nearly double the yield obtained by the chromatographic isolation method.

Preparation of 1:2 mAb-CTNNB1 siRNA conjugates. 1:2 mAb-siRNA conjugates were prepared using strain-promoted azide-alkyne cycloaddition chemistry (SPAAC). Azido-modified mAbs (apa or GalNAz as described above) were diluted to 1 mg/mL in DPBS and reacted with 5 equivalents of a chemically stabilized dibenzocyclooctyne (DBCO)-modified siRNA targeting CTNNB1 (DBCO-CTNNB1 siRNA) overnight at 37 °C. The mAb-siRNA conjugates (PSMB127-apa-siRNA degree of labeling (DOL) of 2, Trastuzumab-apa-siRNA DOL 2, and Trastuzumab-GalNAz-siRNA DOL 2) were purified by size exclusion chromatography and analyzed by reduced SDS-PAGE and analytical size exclusion chromatography.

Preparation of 1:1 PSMB127-CTNNB1 siRNA conjugate by controlled Fab-arm exchange method. IgG4 mAbs can exchange half-molecules with other IgG4 mAbs due to their flexible hinge regions and somewhat destabilized CH3 domains. This process can be promoted by combining a wild-type IgG4 mAb with a mutant mAb containing two point mutations (F405L/R409K); the formation of the resultant heterodimer is energetically preferred over the combination of either homodimer. This method of controlled Fab-arm exchange was used to generate PSMB127 containing a single azide moiety for subsequent SPAAC conjugation of siRNA to yield a 1:1 mAb-siRNA conjugate. PSMB127-apa (1 mg/mL) was combined with PSMB127 F405L/R409K (1 mg/mL) in the presence of 50 mM cysteamine. The reaction was incubated overnight at 37 °C. After the fab-arm exchange reaction was complete, the resultant bi-specific mAb (PSMB127-apa DOL 1) was exchanged into DPBS using a desalting column and allowed to re-oxidize at 4 °C for 2 days. Next, PSMB 127-apa DOL 1 was reacted with 5 equivalents of a DBCO-CTNNB1 siRNA overnight at 37 °C. The 1:1 mAb-siRNA conjugate (PSMB127-apa-siRNA cFAE DOL 1) was purified by size exclusion chromatography and analyzed by reduced SDS-PAGE and analytical size exclusion chromatography.

Preparation of 1:1 Trastuzumab-CTNNB1 siRNA conjugates by chromatographic isolation method. Trastuzumab-GalNAz (1 mg/mL) was reacted with 1.5 equivalents of DBCO-CTNNB1 siRNA overnight at 37 °C. The resultant crude reaction mixture contained 1:0, 1:1, and 1:2 Trastuzumab-siRNA conjugates. To isolate the 1:1 Trastuzumab-siRNA conjugate, the reaction mixture was purified over a TSK-Gel™ BioAssist™ Q column (Tosoh). Chromatographic fractions containing the 1:1 Trastuzumab-siRNA conjugate (Trastuzumab-GalNAz-siRNA DOL 1) were combined and exchanged into DPBS using a desalting column. Trastuzumab-GalNAz-siRNA DOL 1 was analyzed by reduced SDS-PAGE and analytical size exclusion chromatography. Trastuzumab-apa-siRNA DOL 1 was prepared in a similar manner as described above, except only 1 equivalent of DBCO-CTNNB1 siRNA was used to generate the crude reaction mixture. The obtained yields of the 1:1 mAb-siRNA conjugates (from each respective azido-mAb intermediate) were approximately 30% in each case.

### Example 2

### Thermal Stability Analysis of PSMB127-siRNA Conjugates.

PSMB127-siRNA conjugates were characterized more fully to assess the impact of the siRNA conjugation strategy on the thermal stability of the mAb. Melting temperature (Tₘ) was determined by differential scanning fluorimetry using a nanoDSF instrument from Nanotemper. Tₘ was determined by monitoring changes in fluorescence intensity at 330 and 350 nm upon thermal scanning from 20 to 95 °C. While a lower Tₒₙₛₑₜ (start of unfolding) was observed for the modified PSMB 127 variants, the Tₘ was not significantly impacted by conjugation to siRNA. Notably, the Tₘ of both 1:1 mAb-siRNA conjugates generated by the herein disclosed methods or by cFAE (PSMB127-GalNAz-siRNA DOL 1 and PSMB127-apa-siRNA cFAE DOL 1) were similar, demonstrating that the disclosed methods do not grossly impact the thermal stability the parental mAb.

TABLE 1
Thermal stability analysis of PSMB 127-siRNA conjugates.

| Construct | Tₒₙₛₑₜ (°C) | Tₘ₁ (°C) |
|---|---|---|
| PSMB127 | 59.9 | 65.3 |
| PSMB127-apa | 48.7 | 63.5 |
| PSMB127-GalNAz | 52.3 | 63.3 |
| PSMB127-GalNAz-siRNA stitched DOL 1 | 56.0 | 64.3 |
| PSMB127-apa-siRNA cFAE DOL 1 | 53.5 | 63.8 |
| PSMB127-apa siRNA DOL 2 | 47.1 | 63.4 |

Target mRNA Knockdown after mAb-Mediated Uptake of siRNA.

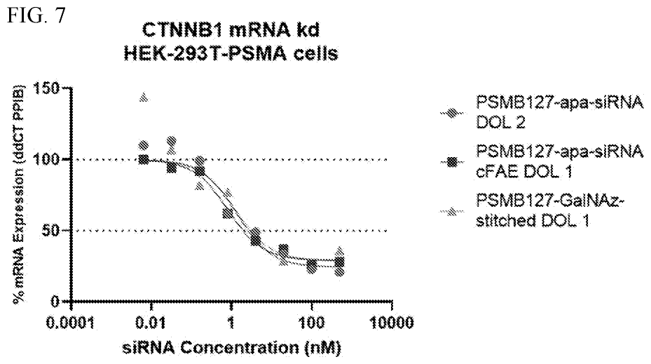

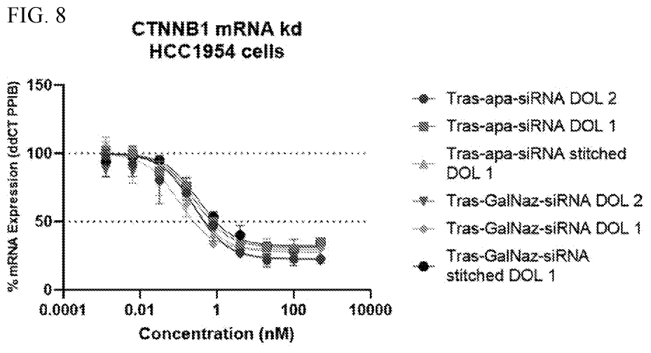

PSMB127 and Trastuzumab-siRNA conjugates were assessed for their ability to mediate knockdown of CTNNB1 mRNA. PSMB127-siRNA conjugates were evaluated in stably transfected prostate-specific membrane antigen (PSMA)-expressing HEK-293T cells and Trastuzumab-siRNA conjugates were evaluated in HCC1954 cells. Cells were treated with siRNA conjugates for 72 hours in the absence of transfection reagent. The extent of mRNA knockdown was determined using real-time quantitative PCR (RT-qPCR) (FIG. 7 and FIG. 8). The PSMB 127 and Trastuzumab-siRNA conjugates were equally potent in each respective cell line. Taken together, these data demonstrate that the disclosed methods have no discernable impact on mAb uptake and siRNA delivery when directly compared to traditionally prepared 1:1 or 1:2 mAb-siRNA conjugates.

### Pharmacokinetic Evaluation of Trastuzumab-siRNA Conjugates in Mice.

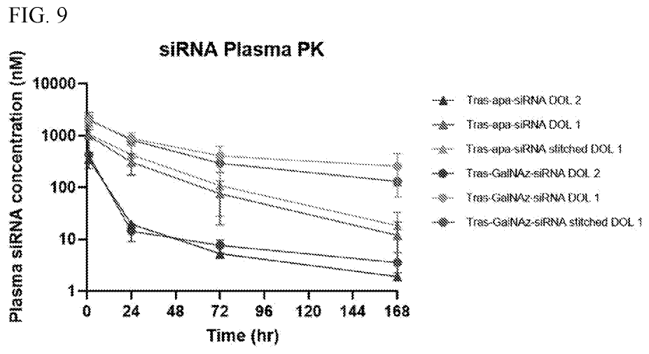

To assess the pharmacokinetic properties of the Trastuzumab-siRNA conjugates, C57BL.6 mice (n=3 per group) were intravenously dosed with each conjugate at 1 mg/kg (siRNA). Blood samples were collected after 1, 24, 72, and 168 hours following the single dose. Immediately after collection, the blood samples were centrifuged, and the resultant plasma was frozen at −80 °C until further analysis. The plasma concentration of CTNNB1 siRNA at each time point was determined using stem-loop RT-qPCR methods (FIG. 9). Overall, the 1:1 Trastuzumab-siRNA conjugates exhibited significantly higher plasma concentrations at each time point compared to the 1:2 Trastuzumab-siRNA conjugates (Table 2). Unexpectedly, the total in vivo exposures observed for the 1:1 GalNAz-siRNA conjugates were significantly higher (~2-4 fold) than those observed for the 1:1 apa-siRNA conjugates.

TABLE 2
Mean siRNA plasma concentration (nM) and total siRNA exposure (nmol

| | | | Tras-apa- | | | Tras-GalNAz- |
|---|---|---|---|---|---|---|
| time | Tras-apa- | Tras-apa- | siRNA DOL 1, | Tras-GalNAz- | Tras-GalNAz- | siRNA DOL 1, |
| (hr) | siRNA DOL 2 | siRNA DOL 1 | stitched | siRNA DOL 2 | siRNA DOL 1 | stitched |
| | mean siRNA plasma concentration (nM) | | | | | |
| 1 | 350 ± 69.6 | 993.0 ± 324.9 | 1068.7 ± 375.3 | 424.5 ± 262.6 | 1949.0 ± 292.5 | 2053.9 ± 430.6 |
| 24 | 19.6 ± 0.55 | 307.0 ± 78.2 | 429.1 ± 145.2 | 14.3 ± 3.0 | 881.4 ± 147.1 | 814.2 ± 84.2 |
| 72 | 5.3 ± 0.58 | 75.5 ± 32.7 | 109 ± 46.7 | 7.6 ± 1.2 | 405.7 ± 124.7 | 292.1 ± 56.2 |
| 168 | 1.9 ± 0.17 | 11.9 ± 5.53 | 18.2 ± 8.6 | 3.5 ± 1.1 | 255.8 ± 110.4 | 129.8 ± 13.0 |
| | AUC (nmol siRNA*day/L) | | | | | |
| | 159.5 ± 17.6 | 1039.0 ± 232.2 | 1326.8 ± 225.9 | 171.7 ± 72.2 | 3868.8 ± 897.8 | 3173.5 ± 426.9 |

siRNA*day/L) following treatment with a single dose (1 mg/kg siRNA) of each Trastuzumab-siRNA conjugate.

1:1 mAb-siRNA conjugates generated by the disclosed methods retained favorable biophysical stability profiles and induced efficient gene silencing equivalent to 1:1 mAb-siRNA conjugates generated by other methods, such as chromatographic isolation. Furthermore, pharmacokinetic (PK) studies in mice revealed that the in vivo exposures of the mAb-siRNA conjugates generated by the disclosed methods are comparable to those 1:1 mAb-siRNA conjugates generated by other methods and are substantially improved relative to analogous 1:2 mAb-siRNA controls. Taken together, the disclosed methods of generating 1:1 mAb-siRNA conjugates provides significant improvements in synthetic strategy and yield while maintaining the potency of 1:1 mAb-siRNA conjugates prepared by other methods, and dramatically improves circulation pharmacokinetics compared to 1:2 mAb-siRNA conjugates.

Those skilled in the art will appreciate that numerous changes and modifications can be made to the preferred embodiments disclosed herein and that such changes and modifications can be made without departing from the spirit of the invention. It is, therefore, intended that the appended claims cover all such equivalent variations as fall within the true spirit and scope of the invention.

We claim:

1. A method of forming an antibody-polynucleotide conjugate, the method comprising
contacting:
(a) an antibody comprising two reactive bioorthogonal groups; and
(b) a polynucleotide comprising a bifurcated linker that comprises two reactive groups,
wherein the reactive groups on the bifurcated linker selectively interact with the biorthogonal groups to form a conjugate having an antibody-polynucleotide ratio that is about 1:1.

2. The method of claim 1, wherein the polynucleotide is DNA or RNA.

3. The method of claim 2, wherein the DNA or the RNA is a phosphorodiamidate morpholino oligomer.

4. The method of claim 2, wherein the RNA is an siRNA, a miRNA, a gRNA, shRNA, or an antisense oligonucleotide.

5. The method of claim 1, wherein one antibody heavy chain constant region comprises one reactive biorthogonal group and the other antibody heavy chain constant region comprises the other reactive biorthogonal group.

6. The method of claim 1, wherein each of the two reactive biorthogonal groups comprises an azide group.

7. The method of claim 6, wherein at least one of the azide groups is selected from the group consisting of 3-azidopropylamine, N-azidoacetylgalactosamine, 6-azido GalNAc, azido-butylamine, azidepentylamine, and azide-PEG3-amine.

8. The method of claim 7, wherein the antibody has a 3-azidopropylamine at amino acid position Q295.

9. The method of claim 7, wherein the antibody has a N-azidoacetylgalactosamine at amino acid position N297.

10. The method of claim 1, wherein the bifurcated linker comprises two alkyne groups.

11. The method of claim 10, wherein the alkyne group comprises a cyclooctyne.

12. The method of claim 10, wherein the bifurcated linker is a Mal-PEG(X)-bis-PEG(X)-BCN linker or a Mal-PEG(X)-bis-PEG(X)-DBCO linker.

13. The method of claim 12, wherein the bifurcated linker is a Mal-PEG2-bis-PEG3-BCN.

14. (canceled)

15. (canceled)

16. (canceled)

17. The method of claim 1, wherein the antibody-polynucleotide conjugate is produced at an overall yield of about 55% to about 70%.

18. The method of claim 1, wherein the antibody-polynucleotide conjugate has an improved circulation pharmacokinetic (PK) profile over an antibody-polynucleotide conjugate that has a 1:2 ratio.

19. A method of forming an antibody-polynucleotide conjugate, the method comprising contacting an antibody and a polynucleotide,
wherein
(a) each antibody heavy chain constant region comprises an azide group; and
(b) the polynucleotide comprises a bifurcated linker that comprises two alkyne groups,
wherein each azide group reacts with an alkyne group to form a conjugate having an antibody-polynucleotide ratio that is about 1:1.

20. The method of claim 19, wherein the polynucleotide is DNA or RNA.

21. The method of claim 20, wherein the DNA or the RNA is a phosphorodiamidate morpholino oligomer.

22. The method of claim 20, wherein the RNA is an siRNA, a miRNA, a gRNA, shRNA, or an antisense oligonucleotide.

23. The method of claim 18, wherein at least one of the azide groups is selected from the group consisting of 3-azidopropylamine, N-azidoacetylgalactosamine, 6-azido GalNAc, azido-butylamine, azidepentylamine, and azide-PEG3-amine.

24. The method of claim 23, wherein the antibody has a 3-azidopropylamine at amino acid position Q295.

25. The method of claim 23, wherein the antibody has a N-azidoacetylgalactosamine at amino acid position N297.

26. The method of claim 1, wherein the alkyne group comprises a cyclooctyne.

27. The method of claim 18, wherein the bifurcated linker is a Mal-PEG(X)-bis-PEG(X)-BCN or a Mal-PEG(X)-bis-PEG(X)-DBCO.

28. The method of claim 27, wherein the bifurcated linker is a Mal-PEG2-bis-PEG3-BCN.

29. (canceled)

30. (canceled)

31. (canceled)

32. (canceled)

33. (canceled)

34. (canceled)

35. (canceled)

36. A polynucleotide-conjugated antibody comprising:
a polynucleotide attached to a bifurcated linker; and
an antibody comprising two heavy chain constant regions that each comprise a bioorthogonal group;
wherein the bifurcated linker is attached to the bioorthogonal groups of each heavy chain constant region.

37. The polynucleotide-conjugated antibody of claim 36, wherein the polynucleotide is DNA or RNA.

38. The polynucleotide-conjugated antibody of claim 37, wherein the DNA or the RNA is a phosphorodiamidate morpholino oligomer.

39. The polynucleotide-conjugated antibody of claim 37, wherein the RNA is an siRNA, a miRNA, a gRNA, shRNA, or an antisense oligonucleotide.

40. The polynucleotide-conjugated antibody of claim 36, wherein the polynucleotide is conjugated to the antibody by a triazole.

41. The polynucleotide-conjugated antibody of claim 36, wherein the bifurcated linker is a Mal-PEG(X)-bis-PEG(X)-BCN or a Mal-PEG(X)-bis-PEG(X)-DBCO.

42. The polynucleotide-conjugated antibody of claim 41, wherein the bifurcated linker is a Mal-PEG2-bis-PEG3-BCN.

43. (canceled)

44. (canceled)

45. (canceled)

46. (canceled)

47. A method of forming an antibody-polynucleotide conjugate, the method comprising contacting:
(a) an antibody comprising a F405L substitution and a R409K substitution on one heavy chain constant region and an azide group on the other heavy chain constant region; and
(b) a polynucleotide;
to form a conjugate having an antibody-polynucleotide ratio that is about 1:1.

48. A method of promoting Fab arm exchange comprising contacting:
(a) a first antibody comprising a F405L substitution and a R409K substitution; and
(b) a second antibody that does not have the F405L substitution and the R409K substitution and that comprises an azide group on one or both of the antibody heavy chain constant regions,
to form an antibody comprising the F405L substitution and the R409K substitution on one heavy chain constant region and the azide group on the other heavy chain constant region.

49. The method of claim 47, wherein the azide group on one or both of the antibody heavy chain constant regions is selected from 3-azidopropylamine, N-azidoacetylgalactosamine, 6-azido GalNAc, azido-butylamine, azidepentylamine, or azide-PEG3-amine.

50. The method of claim 49, wherein the azide group is a 3-azidopropylamine at amino acid position Q295.

51. The method of claim 49, wherein the azide group is a N-azidoacetylgalactosamine at amino acid position N297.

52. The method of claim 47, wherein the antibody comprising the F405L substitution and the R409K substitution on one heavy chain constant region and the azide group on the other heavy chain constant region is a bispecific antibody.

53. The method of claim 48, wherein the azide group on one or both of the antibody heavy chain constant regions is selected from 3-azidopropylamine, N-azidoacetylgalactosamine, 6-azido GalNAc, azido-butylamine, azidepentylamine, or azide-PEG3-amine.

54. The method of claim 53, wherein the azide group is a 3-azidopropylamine at amino acid position Q295.

55. The method of claim 53, wherein the azide group is a N-azidoacetylgalactosamine at amino acid position N297.
